(19) United States
(12) Patent Application Publication
(10) Pub. No.: US 2010/0137702 A1
(43) Pub. Date: Jun. 3, 2010

(54) APPARATUS FOR MEASURING PHYSIOLOGICAL SIGNAL OF VEHICLE DRIVER

(75) Inventors: Seon Hee PARK, Daejeon (KR); Seung Hwan KIM, Daejeon (KR); In Bum LEE, Daejeon (KR); Seung Chul SHIN, Daejeon (KR); Yong Won JANG, Daejeon (KR); Yoon Seon SONG, Daejeon (KR)

Correspondence Address: LADAS & PARRY LLP, 224 SOUTH MICHIGAN AVENUE, SUITE 1600, CHICAGO, IL, 60604, US

(73) Assignee: ELECTRONICS AND TELECOMMUNICATIONS RESEARCH INSTITUTE, Daejeon (KR)

(21) Appl. No.: 12/629,341

(22) Filed: Dec. 2, 2009

(30) Foreign Application Priority Data
Dec. 3, 2008 (KR) 10-2008-0121855
Nov. 18, 2009 (KR) 10-2009-0111260

(51) Int. Cl. A61B 5/04; A61B 5/0402

(52) U.S. Cl. 600/393; 600/509

(57) ABSTRACT

An apparatus for measuring a physiological signal of a vehicle driver includes: a conductive fabric electrode coated on a target object within a vehicle and detecting a physiological current of a driver when it comes in contact with the driver; and a signal generating unit generating an electrical signal corresponding to the physiological current.

### CROSS-REFERENCE TO RELATED APPLICATIONS

This application claims the priorities of Korean Patent Application Nos. 10-2008-0121855 filed on Dec. 3, 2008, and 10-2009-0111260 filed on Nov. 18, 2009 in the Korean Intellectual Property Office, the disclosures of which are incorporated herein by reference.

### BACKGROUND OF THE INVENTION

1. Field of the Invention

The present invention relates to an apparatus for measuring a physiological signal (i.e., biological signal, bio signal, etc.) of a vehicle driver and, more particularly, to a technique that measures a physiological signal of a vehicle driver without the driver's awareness when the driver's hand comes into contact with a fabric electrode sensor which is coated on a location with which the driver's hand comes into contact within a vehicle.

2. Description of the Related Art

In general, monitoring the condition of a driver's heart while he is driving is meaningful in that a safe driving is actively induced as well as in terms of home healthcare aimed at observing the driver's physical condition in the long term.

Recently, a great deal of research and development into a technique of measuring physiological signals of vehicle drivers has been conducted. In this case, it is desirous to measure a physiological signal regarding the driver's physical condition in a state wherein the driver is not interfered with by the measurement and is unaware that measurement is being made. It would also be desirous to do research and development in this direction.

### SUMMARY OF THE INVENTION

An aspect of the present invention provides an apparatus for measuring a physiological signal of a vehicle driver by coating a conductive fabric electrode on a location with which the driver's hand naturally comes in contact in a vehicle such that it allows for a smooth contact by the driver's hand and provides a satisfactory contact sense of the driver, whereby a physiological signal of the driver can be measured while the driver is unaware of measurement being made.

According to an aspect of the present invention, there is provided an apparatus for measuring a physiological signal of a vehicle driver, including: a conductive fabric electrode coated on a target object within a vehicle and detecting a physiological current of a driver when it comes in contact with the driver; and a signal generating unit generating an electrical signal corresponding to the physiological current.

According to another aspect of the present invention, there is provided a vehicle steering wheel coated with a first conductive fabric electrode with which the driver's left hand comes in contact and a second conductive fabric electrode, with which the driver's right hand comes in contact, separated from the first conductive fabric electrode, including: a signal generating unit detecting a physiological current of the driver through the first and second conductive fabric electrodes to generate a physiological electrical signal of the driver corresponding to the physiological current.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 illustrates the configuration of an apparatus for measuring a physiological signal according to an exemplary embodiment of the present invention;

FIG. 2 is a schematic block diagram showing the configuration of a fabric electrode and signal generating unit in the physiological signal measurement apparatus according to an exemplary embodiment of the present invention;

FIG. 3 is a schematic block diagram of an electrocardiogram (ECG) measurement device using the apparatus for measuring a physiological signal of a vehicle driver according to an exemplary embodiment of the present invention;

FIG. 4 is a development view of a conductive fabric electrode and a packing material of a wheel frame in the physiological signal measurement apparatus according to an exemplary embodiment of the present invention;

FIG. 5 illustrates the thread of first and second textiles of the conductive fabric electrode in the physiological signal measurement apparatus according to an exemplary embodiment of the present invention;

FIG. 6 illustrates a filler insertedly positioned between the conductive fabric electrode and the wheel frame in the physiological signal measurement apparatus according to another exemplary embodiment of the present invention;

FIG. 7 is a schematic block diagram of an apparatus for measuring a physiological signal additionally including an adaptive filter unit according to another exemplary embodiment of the present invention;

FIG. 8 is a schematic block diagram showing the configuration of the adaptive filter unit used for the physiological signal measurement apparatus according to another exemplary embodiment of the present invention; and

FIG. 9 is a schematic block diagram of an apparatus for measuring a physiological signal additionally including a transmission unit according to another exemplary embodiment of the present invention.

### DETAILED DESCRIPTION OF THE PREFERRED EMBODIMENT

Exemplary embodiments of the present invention will now be described in detail with reference to the accompanying drawings. The invention may however be embodied in many different forms and should not be construed as limited to the embodiments set forth herein. Rather, these embodiments are provided so that this disclosure will be thorough and complete, and will fully convey the scope of the invention to those skilled in the art. In the drawings, the shapes and dimensions may be exaggerated for clarity, and the same reference numerals will be used throughout to designate the same or like components.

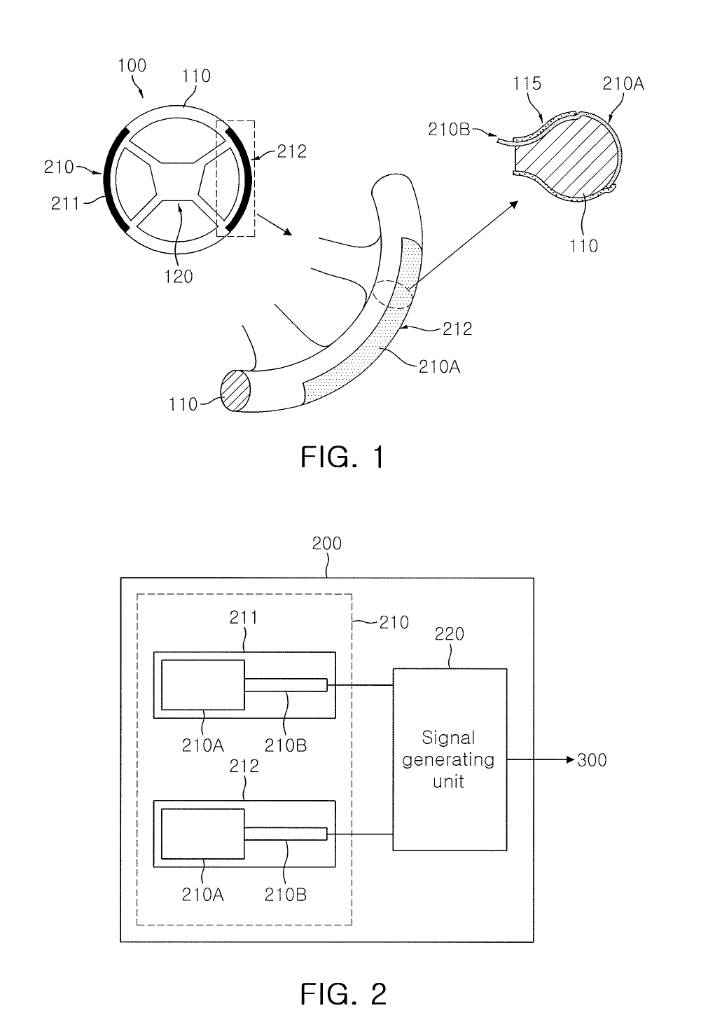

FIG. 1 illustrates the configuration of an apparatus for measuring a physiological signal according to an exemplary embodiment of the present invention. FIG. 2 is a schematic block diagram showing the configuration of a fabric electrode and signal generating unit in the physiological signal measurement apparatus according to an exemplary embodiment of the present invention.

With reference to FIGS. 1 and 2, an apparatus for measuring a physiological signal according to an exemplary embodiment of the present invention may include a conductive fabric electrode 210 and a signal generating unit 220.

The conductive fabric electrode 210, which is coated on a wheel frame to detect a physiological current of a driver when the driver's body comes in contact therewith, may include a first conductive fabric electrode 211 installed at a left portion of a steering wheel of the wheel frame 110 so as to be in contact with the driver's left hand and a second conductive fabric electrode 212 electrically separated from the first conductive fabric electrode 211 and installed at a right portion of the steering wheel of the wheel frame 110 so as to be in contact with the driver's right hand.

Each of the conductive fabric electrodes 211 and 212 may include a contact portion 210A coated on the wheel frame 110 and exposed to be in contact with the driver's hand and a connection portion 210B connected with the contact portion 210A and formed between a packing material 115 coated on the wheel frame 110 and the wheel frame 110 so as to be electrically connected with a signal generating unit 220.

In this manner, in the physiological signal measurement apparatus according to the present exemplary embodiment of the present invention, the conductive fabric electrode 210 and the signal generating unit 220 can be connected by means of the connection portion 210B extending from the contact portion 210A of the conductive fabric electrode 210, rather than by a wire.

The location of the signal generating unit 220 is not illustrated in FIG. 1. The signal generating unit 220 may be installed at a central portion 120 of the steering wheel 100.

As described above, the first and second fabric electrodes 211 and 212 are coated on the sufficiently large area in a state of being electrically separated at both sides of the wheel frame 110 of the steering wheel 100, whereby even when the locations of the vehicle driver's both hands change, a physiological signal of the driver can be measured. Namely, when the vehicle driver holds the both sides of the wheel frame 110 of the steering wheel 100, the driver's both hands come in contact with the fabric electrodes 210 coated on both sides of the wheel frame 100, and accordingly, the signal generating unit 220 can generate an electrical signal corresponding to the physiological current of the driver.

In the present exemplary embodiment, the conductive fabric electrode 210 is coated on the wheel frame 110 of the steering wheel 100, but the target subjected to coating of the conductive fabric electrode 210 is not limited thereto. Namely, for example, the conductive fabric electrode 210 can be coated on any location within the vehicle so long as the driver's hand can come in contact therewith and the contact state is maintained for a certain time duration, such as on the gear stick of the vehicle, the window frame or the arm beside a driver's seat, and the like.

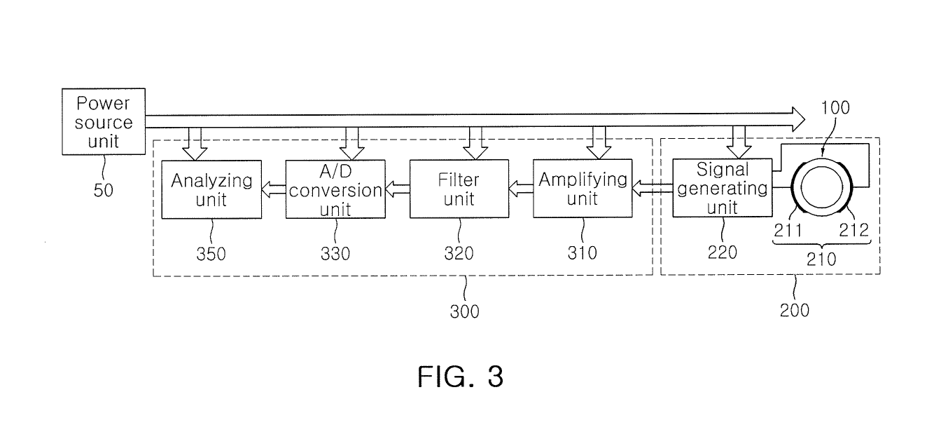

FIG. 3 is a schematic block diagram of an electrocardiogram (ECG) measurement device using the apparatus for measuring a physiological signal of a vehicle driver according to an exemplary embodiment of the present invention.

With reference to FIG. 3, the device for measuring a vehicle driver's electrocardiogram according to the present exemplary embodiment may include a fabric electrode sensor 200 including the conductive fabric electrode 210 coated on the wheel frame 110 of the steering wheel 100 of the vehicle to detect a physiological current of the driver and the signal generating unit 220 generating an electrocardiogram signal corresponding to the driver's physiological current detected through the conductive fabric electrode 210, and an electrocardiogram processor 300 processing the electrocardiogram signal received from the fabric electrode sensor 200.

The device for measuring the vehicle driver's electrocardiogram includes a power source unit 50. The power source unit 50 may use a power source of the vehicle (i.e., vehicle battery, etc.) or any other power source, and regulate the power provided to have a required voltage and supply the same.

The electrocardiogram processor 300 may include an amplifying unit 310 amplifying the electrocardiogram signal transferred from the fabric electrode sensor 210; a filter unit 320 canceling noise included in the electrocardiogram signal transferred from the amplifying unit 310; an A/D conversion unit 330 converting the electrocardiogram signal transferred from the filter unit 320 into a digital signal; and an analyzing unit 350 analyzing the digital electrocardiogram signal transferred from the A/D conversion unit 330.

The amplifying unit 310 amplifies the electrocardiogram signal transferred from the signal generating unit 220. For example, the electrocardiogram signal with the magnitude of substantially 1 mV measured through the first and second conductive fabric electrodes 211 and 212 of the fabric electrode sensor 210 is differentially amplified at a pre-set amplification degree (e.g., the amplification degree is 1000 times) by the amplifying unit 310 and then outputted to the filter unit 320.

The filter unit 320 cancels noise included in the electrocardiogram signal transferred from the amplifying unit 310 and outputs the noise-canceled electrocardiogram signal to the A/D conversion unit 330. Here, for example, the filter unit 320 may allow the portion of the electrocardiogram signal corresponding to a frequency bandwidth ranging from 0.01 Hz to 100 Hz to pass therethrough, while canceling noise of other frequency bands.

The A/D conversion unit 330 converts the electrocardiogram signal transferred from the filter unit 320 into a digital signal and outputs the digital signal to the analyzing unit 350. Namely, the A/D conversion unit 330 may binarize the analog electrocardiogram signal which has passed through the filter unit 320 to convert it into the digital electrocardiogram signal (e.g., 12 bits, 200 Hz), and output the same.

The analyzing unit 350 analyzes the digital electrocardiogram signal transferred from the A/D conversion unit 330 to analyze the vehicle driver's electrocardiogram.

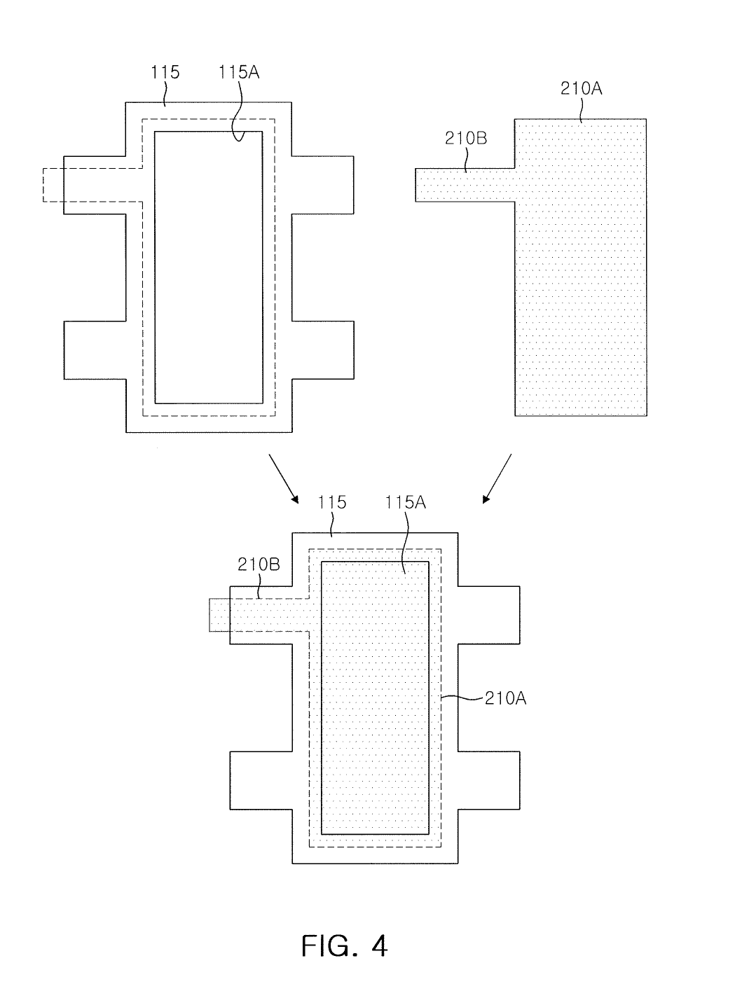

FIG. 4 is a development view of a conductive fabric electrode and a packing material of a wheel frame in the physiological signal measurement apparatus according to an exemplary embodiment of the present invention.

With reference to FIG. 4, in the physiological signal measurement apparatus according to the present exemplary embodiment, the conductive fabric electrode 210 includes the contact portion 210A and the connection portion 210B as mentioned above. In the case where the conductive fabric electrode 210 is coated on the wheel frame 110 and the packing material 115 is coated on the conductive fabric electrode 210, the packing material 115 may have an opening 115A formed at a certain portion of the packing material 115 in order to allow the conductive fabric electrode 210 to be exposed so as to be in contact with the driver's hand.

Accordingly, the other portions of the wheel frame 110, which are not coated by the conductive fabric electrode, may be coated with the packing material 115 made of a leather material used for the wheel frame 110, and in this case, as shown in FIGS. 1 to 4, a portion of the conductive fabric electrode 210 is also covered by the packing material 115, which is thus not seen by the driver.

For example, the packing material 115 includes the opening 115A formed by boring the middle portion, so when the packing material 115 and the conductive fabric electrode 210 are coated in the overlap manner, the dotted line of the packing material 115 in FIG. 4 represents the edges of the conductive fabric electrode 210.

In addition, the conductive fabric electrode 210 may be formed in various manners by giving conductivity to woven textiles.

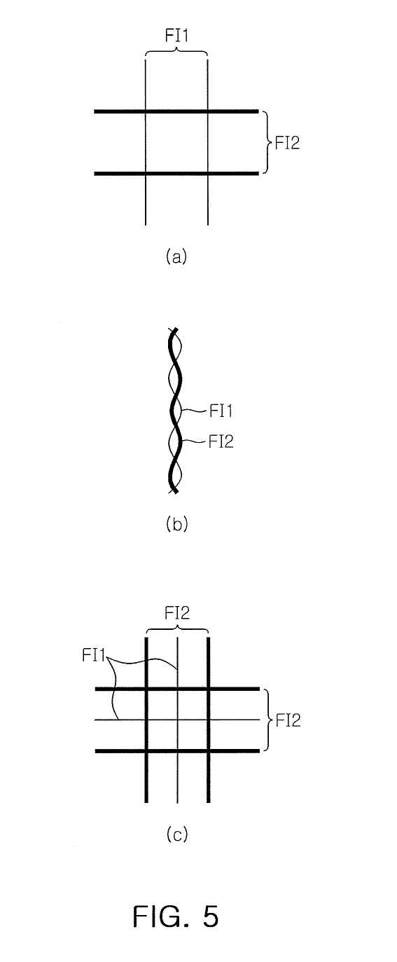

FIG. 5 illustrates the thread of first and second textiles of the conductive fabric electrode in the physiological signal measurement apparatus according to an exemplary embodiment of the present invention.

With reference to FIG. 5, the conductive fabric electrode 210 may be woven by using a general textile and a conductive textile.

For example, in order to weave the conductive fabric electrode 210, various methods such as a method of weaving polyester thread (yarn) with a filament or staple structure and a fiber (textile) coated with a metal such as silver which does not cause a trouble to the skin and has good electrical conductivity according to a tricot or knit, or knitting method, to maintain a proper elasticity.

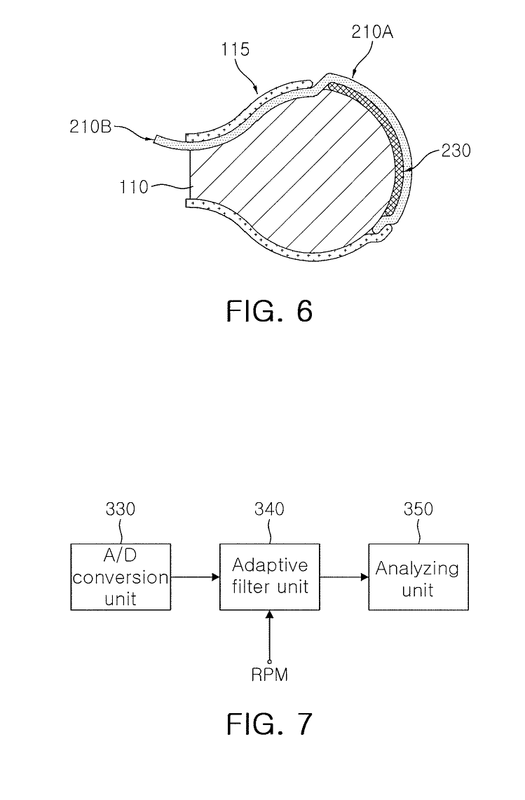

FIG. 6 illustrates a filler insertedly positioned between the conductive fabric electrode and the wheel frame in the physiological signal measurement apparatus according to another exemplary embodiment of the present invention.

With reference to FIG. 6, the physiological signal measurement apparatus may further include an elastic filler 230 in order to improve a contact sense when the driver comes in contact with the conductive fabric electrode. Namely, the filter 230 may be inserted between the wheel frame 110 and the conductive fabric electrode 210 to allow the driver's hand to be in smooth contact with the conductive fabric electrode 210.

With the elastic filler 230, the contact between the wheel frame 110 of the steering wheel 110 and the driver's palm can be smoothly made.

FIG. 7 is a schematic block diagram of an apparatus for measuring a physiological signal additionally including an adaptive filter unit according to another exemplary embodiment of the present invention.

With reference to FIG. 7, an electrocardiogram processor according to the present exemplary embodiment may further include an adaptive filter unit 340 between the A/D conversion unit 330 and the analyzing unit 350, and in this case, the adaptive filter unit 340 may cancel background noise (v) from the digital electrocardiogram signal transferred from the A/D conversion unit 330.

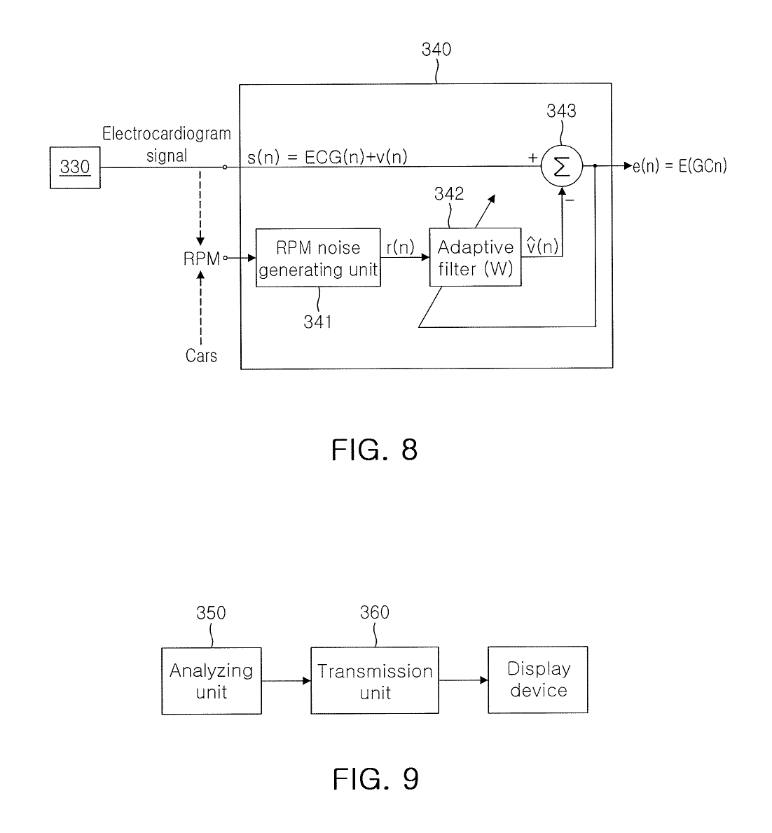

FIG. 8 is a schematic block diagram showing the configuration of the adaptive filter unit used for the physiological signal measurement apparatus according to another exemplary embodiment of the present invention.

With reference to FIG. 8, the adaptive filter unit 340 may include a revolutions per minute (RPM) noise generating unit 341, an adaptive filter 342, and a subtracter 343.

The RPM noise generating unit 341 may generate an RPM noise signal (r) corresponding to an RPM of an engine of the vehicle, and output it to the adaptive filter 342.

In order to reduce background noise included in the digital electrocardiogram signal transferred from the A/D conversion unit 330, the adaptive filter 342 may generate a virtual background noise (v) by means of the RPM noise signal (r) transferred from the RPM noise generating unit 341 and output the generated virtual background noise (V) to the subtracter 343. The subtracter 343 then subtracts the virtual background noise transferred from the adaptive filter 342, from the digital electrocardiogram signal transferred from the A/D conversion unit 330. Through this process, the background noise included in the electrocardiogram signal can be canceled.

Namely, the electrocardiogram signal has the background noise (v) generated by the vehicle it is placed therein. In this case, the background noise (v) is vibration and electrical noise mainly generated according to the RPM of the engine, having the same frequency characteristics as the RPM of the engine. For example, if the engine revolves at 3000 rpm, background noise of 50 Hz is mixed in the electrocardiogram signal and received. The RPM of the engine can be provided by the vehicle, or otherwise, it can be discovered by analyzing the frequency of the measured electrocardiogram signal. When the RPM of the engine is given, background noise (v) can be canceled by using the adaptive filter unit 340.

Here, the adaptive filter unit 340 is implemented as an adaptive filter adjusting the power of virtual background noise according to the cancellation degree of the background noise included in the electrocardiogram signal, instead of using a band stop filter having a fixed pass band. Thus, the adaptive filter unit 340 can effectively cancel the background noise caused by the revolution of the varying engine.

Accordingly, quality of the binarized electrocardiogram (ECG) signal input to the analyzing unit 350 may be improved while the signal passes through the adaptive filter unit 340.

For example, if the engine is driven at 3000 rpm, the RPM noise generating unit 341 generates RPM noise, a sine wave of 3000/60=50 Hz. The generated RPM noise passes through the adaptive filer 342 to implement virtual background noise (rmhatv) corresponding to the noise background (v) mixed in the measured electrocardiogram signal. Thereafter, the subtracter 343 subtracts background noise from the electrocardiogram signal and outputs the background nose-canceled electrocardiogram signal. The adaptive filter 342 may be updated to minimize the background noise of the electrocardiogram signal.

Here, in order to quickly cope with the change in the RPM of the engine, the adaptive filter may be updated by using a recursive least square (RLS), instead of a least mean square (LMS).

In an exemplary embodiment of a noise canceling algorithm using the RLS method, as shown in FIGS. 6 and 7, if an objective function for minimizing [symbol] (n) output from the adaptive filter unit 340 according to the exemplary embodiment of the present invention is defined as

$${}^{‵} \hat{ECG} ( n ) = {\sum }_{i = 0}^{n} {ECG ( i )}^{2} ,$$

in case where the adaptive filter 342 according to an exemplary embodiment of the present invention is designed as a finite impulse response (FIR) filter, a method of updating a weight value of the adaptive filter 342 is carried out as follows.

Step 1: The background noise-canceled electrocardiogram signal

$$( \hat{ECG} ( \frac{n}{n - 1} ) )$$

is calculated as represented by Equation 1 shown below by using the signal (s(n)) transferred from the A/D conversion unit 330, a pre-set previous weight value (w(n−1)), and the RPM noise signal (r) transferred from the RPM noise generating unit 341:

$$\hat{ECG} ( \frac{n}{n - 1} ) = s ( n ) - {\overline{w} ( n - 1 )}^{T} \overline{r} ( n ) \qquad [ Equation 1 ]$$

Step 2: A gain vector ( g̅(n)) is calculated as represented by Equation 2 shown below by using an inverse matrix of an initial unit matrix (R(n)) and the RPM noise signal (r):

g̅(n)=R(n)⁻¹ r̅(n) [Equation 2]

Step 3: IR(n) (=R(n)⁻¹) is calculated as represented by Equation 3 shown below by using a previous unit matrix (an inverse matrix (IR(n−1) of R(n−1)) and the RPM noise signal (r):

$$IR ( n ) = \frac{1}{λ} [ IR ( n - 1 ) - \frac{\{ IR ( n - 1 ) {\overline{r} ( n )}^{T} IR ( n - 1 ) \}}{λ + {\overline{r} ( n )}^{T} IR ( n - 1 ) \overline{r} ( n ) \}} ] \qquad [ Equation 3 ]$$

Step 4: A previous weight value ( w̅(n−1)) of the adaptive filter is calculated:

$$\overline{w} ( n ) = \overline{w} ( n - 1 ) + IR ( n ) \overline{r} ( n ) \sim \hat{ECG} ( \frac{n}{n - 1} ) \qquad [ Equation 4 ]$$

Step 5: The weight value ( w̅(n)) is calculated.

v̅(n)= w̅(n)^{T} r̅(n) [Equation 5]

Step 6: The background noise-canceled electrocardiogram signal (RMHAT{ECG} (n)) is calculated as represented by Equation 6 shown below by using the signal (s(n)) transferred from the A/D conversion unit 330 and the virtual background noise (rmhatv):

[symbol]

(n)=s(n)−\hat{v}(n) [Equation 6]

Step 7: When next data is received, the process is repeated starting from the step 1.

Here, r̅(n) is a vector of the RPM noise output from the RPM noise generating unit 341, g̅(n) is a vector of the gain of the adaptive filter 342, w̅(n) is a vector of weighting factors of the adaptive filter 342, and T of w̅(n)⁻ means a transposed matrix.

FIG. 9 is a schematic block diagram of an apparatus for measuring a physiological signal additionally including a transmission unit according to another exemplary embodiment of the present invention.

As shown in FIG. 9, in the apparatus for measuring a physiological signal of a vehicle driver according to the present exemplary embodiment, the processor may further include a transmission unit 400, and in this case, the transmission unit 400 may transfer the information obtained by analyzing the physiological signal by the analyzing unit 350 of the processor to an external device.

Here, the external device may be an external display device such as a navigator or the like. For example, when a navigator is connected to the transmission unit 400, the measured physiological signal of the driver according to an exemplary embodiment of the present invention can be output to a screen via the navigator. According to the present exemplary embodiment, the physiological signal of the driver can be measured and analyzed, and its results can be output, while the driver is not aware of it during driving. Namely, the analysis results output from the physiological signal measurement apparatus can be output to a screen display device or a navigation system mounted in the vehicle for user recognition.

As described above, because the apparatus for measuring a physiological signal of a vehicle driver employs the fabric electrode sensor made of the conductive fabric, the sensor can be coated on a larger portion of the wheel frame, allowing for smooth contact between the driver's hand and the fabric electrode sensor and bettering the user's tactile sensation (impression).

As set forth above, according to exemplary embodiments of the invention, the conductive fabric electrode coated on the wheel frame of the vehicle can ensure contact between the driver's hand and the fabric electrode and better the contact sense of the driver. Thus, the physiological signal of the driver can be measured while the driver is holding the steering wheel but without the driver's recognition. Namely, the physiological signal of the driver can be measured without interfering with the driver who is driving.

That is, because the fabric electrode sensor made of conductive fabric is employed, the fabric electrode can be coated on a large portion of the wheel frame to allow the driver's hand and the conductive fabric electrode to be in contact with each other smoothly and make the driver feel good (namely, improve the tactile impression of the driver). Also, because a portion of the conductive fabric electrode is used as the connection portion, in place of the existing wire, to transfer an electrical signal, the fabrication and construction can be simplified.

In addition, because a band stop filter is implemented by the adaptive filter according to a revolutions per minute (RPM) of the engine, external noise present in the measured physiological signal can be effectively canceled.

While the present invention has been shown and described in connection with the exemplary embodiments, it will be apparent to those skilled in the art that modifications and variations can be made without departing from the spirit and scope of the invention as defined by the appended claims.

What is claimed is:

1. An apparatus for measuring a physiological signal of a vehicle driver, the apparatus comprising:
a conductive fabric electrode coated on a target object within a vehicle and detecting a physiological current of a driver when it comes in contact with the driver; and
a signal generating unit generating an electrical signal corresponding to the physiological current.

2. The apparatus of claim 1, wherein the conductive fabric electrode is coated on at least one or more of a wheel frame of a vehicle steering wheel, a gear stick, an arm beside the driver's seat, and a window frame beside the driver's seat.

3. The apparatus of claim 1, wherein the conductive fabric electrode comprises:
a first conductive fabric electrode with which the driver's left hand comes in contact; and
a second conductive fabric electrode, with which the driver's right hand comes in contact, separated from the first conductive fabric electrode.

4. The apparatus of claim 3, wherein each of the first and second conductive fabric electrodes comprises:
a contact portion exposed to outside, with which the driver's hand comes in contact; and
a connection portion connected with the contact portion and formed between a packing material coated on the target object and the target object so as to be electrically connected with the signal generating unit.

5. The apparatus of claim 1, wherein the conductive fabric electrode is woven with a general textile and a conductive textile having electrical conductivity.

6. The apparatus of claim 1, further comprising:
an elastic filler inserted between the target object and the conductive fabric electrode to allow for a smooth contact between the conductive fabric electrode and the driver's hand.

7. The apparatus of claim 1, further comprising:
a processor processing the electrical signal generated by the signal generating unit.

8. The apparatus of claim 7, wherein the signal generating unit generates an electrocardiogram signal of the driver corresponding to the physiological current, and the processor is an electrocardiogram processor processing the electrocardiogram signal.

9. The apparatus of claim 8, wherein the electrocardiogram processor comprises:
an amplifying unit amplifying the electrocardiogram signal transferred from the signal generating unit;
a filter unit canceling noise included in the electrocardiogram signal transferred from the amplifying unit;
an A/D conversion unit converting the electrocardiogram signal transferred from the filter unit into a digital signal; and
an analyzing unit analyzing the digital electrocardiogram signal transferred from the A/D conversion unit.

10. The apparatus of claim 9, wherein the electrocardiogram processor further comprises:
an adaptive filter unit canceling background noise from the digital electrocardiogram signal transferred from the A/D conversion unit.

11. The apparatus of claim 10, wherein the adaptive filter unit comprises:
a revolutions per minute (RPM) noise generating unit generating an RPM noise signal corresponding to the RPM of an engine of the vehicle
an adaptive filter generating virtual background noise by using the RPM noise signal transferred from the RPM noise generating unit to reduce noise background included in the digital electrocardiogram signal transferred from the A/D conversion unit; and
a subtracter subtracting the virtual background noise transferred from the adaptive filter from the digital electrocardiogram signal transferred from the A/D conversion unit.

12. The apparatus of claim 7, further comprising:
a transmission unit transmitting analysis information of the physiological signal from the processor to an external device.

13. A vehicle steering wheel coated with a first conductive fabric electrode with which the driver's left hand comes in contact and a second conductive fabric electrode, with which the driver's right hand comes in contact, separated from the first conductive fabric electrode, the vehicle steering wheel comprising:
a signal generating unit detecting a physiological current of the driver through the first and second conductive fabric electrodes to generate a physiological electrical signal of the driver corresponding to the physiological current.

14. The vehicle steering wheel of claim 13, wherein each of the first and second conductive fabric electrodes comprises:
a contact portion exposed to outside, with which the driver's hand comes in contact; and
a connection portion connected with the contact portion and formed between a packing material coated on a wheel frame of the vehicle steering wheel and the wheel frame of the vehicle steering wheel so as to be electrically connected with the signal generating unit.

15. The vehicle steering wheel of claim 13, wherein the first and second conductive fabric electrodes are woven with a general textile and a conductive textile having electrical conductivity.

16. The vehicle steering wheel of claim 13, further comprising:
an elastic filler inserted between the wheel frame of the vehicle steering wheel and the conductive fabric electrode to allow for a smooth contact between the conductive fabric electrode and the driver's hand.

17. The vehicle steering wheel of claim 13, wherein the signal generating unit generates an electrocardiogram signal of a driver corresponding to the physiological current, and
the vehicle steering wheel further comprising:
an electrocardiogram processor processing the electrocardiogram signal.

18. The vehicle steering wheel of claim 17, wherein the electrocardiogram processor comprises:
an amplifying unit amplifying the electrocardiogram signal transferred from the signal generating unit;
a filter unit canceling noise included in the electrocardiogram signal transferred from the amplifying unit;
an A/D conversion unit converting the electrocardiogram signal transferred from the filter unit into a digital signal; and
an analyzing unit analyzing the digital electrocardiogram signal transferred from the A/D conversion unit.

19. The apparatus of claim 18, wherein the electrocardiogram processor further comprises:
an adaptive filter unit canceling background noise from the digital electrocardiogram signal transferred from the A/D conversion unit.

20. The apparatus of claim 19, wherein the adaptive filter unit comprises:
an RPM noise generating unit generating an RPM noise signal corresponding to the RPM of an engine of the vehicle;
an adaptive filter generating virtual background noise by using the RPM noise signal transferred from the RPM noise generating unit to reduce noise background included in the digital electrocardiogram signal transferred from the A/D conversion unit; and
a subtracter subtracting the virtual background noise transferred from the adaptive filter from the digital electrocardiogram signal transferred from the A/D conversion unit.
